(19) United States
(12) Patent Application Publication
(10) Pub. No.: US 2006/0286586 A1
(43) Pub. Date: Dec. 21, 2006

(54) Method to diagnose or screen for inflammatory diseases

(75) Inventors: Hemmo Arjan Drexhage, Rotterdam (NL); Cindy Ruwhof, Alphen Aan Den Rijn (NL); Robbert Benner, Barendrecht (NL)

Correspondence Address: TRASK BRITT, P.O. BOX 2550, SALT LAKE CITY, UT, 84110, US

(73) Assignee: Erasmus University Medical Center Rotterdam, Rotterdam (NL)

(21) Appl. No.: 11/446,458

(22) Filed: Jun. 2, 2006

Related U.S. Application Data
(63) Continuation of application No. PCT/NL04/00844, filed on Dec. 3, 2004.

(30) Foreign Application Priority Data
Dec. 4, 2003 (EP) 03078853.3

(51) Int. Cl. C12Q 1/68; C12M 1/34

(52) U.S. Cl. 435/6; 435/287.2

(57) ABSTRACT

The invention relates to the field of medical diagnostics. More specifically, the invention relates to methods to diagnose or screen for inflammatory conditions or disease, including auto-inflammatory disease and affective disorder, in a subject, preferably a human subject, by assaying for a marker for an inflammatory disease. Provided is a method to diagnose, screen for or predict the development of an affective disorder (AD), preferably bipolar disorder (BP), in a subject, the method comprising determining the level of at least one, preferably at least two, more preferably at least three, most preferred at least four, AD-specific gene product(s) in a biological sample isolated from the subject, preferably peripheral blood monocytes, wherein the gene is selected from the group comprising ATF3, phosphodiesterase 4 B, CXCL2, BCL2-related protein A2, Dual specificity phosphatase 2, TNFα-induced protein 3/A20, BTEB1 CXCL3, Chemokine CCL-3 like, CCL-4, CCL20, CX2CR1, Amphiregulin, Thrombomodulin, Heparin-binding EGF-like growth factor, DNA-damaged inducible transcript, V28 chemokine-like receptor, TRAIL. MAPK6, B4BP4, PBEF1, Thrombospondin 1, MAFF, HSP70, CCL2, MCP-3, CCR2, CX3CR1, DOK1, HBB, G-gamma globin, THBD, PHLDA1, DTR and GNLY.

### CROSS-REFERENCE TO RELATED APPLICATIONS

This application is a continuation of PCT International Patent Application No. PCT/NL2004/000844, filed on Dec. 3, 2004, designating the United States of America, and published, in English, as PCT International Publication No. WO 2005/054513 A2, on Jun. 16, 2005, which application claims priority to European Patent Application Serial No. 03078853.3, filed Dec. 4, 2003, the contents of the entirety of each of which is hereby incorporated herein by this reference.

### TECHNICAL FIELD

The invention relates to the field of medical diagnostics. Among others, methods are provided to diagnose or screen for an inflammatory disease, amongst others for an auto-inflammatory disease, by assaying for an inflammatory disease marker.

### BACKGROUND

The immune system produces cytokines and other humoral factors to protect the host when threatened by inflammatory agents, microbial invasion, or injury. In most cases, this complex defense network successfully restores normal homeostasis, but at other times, the immunological mediators may actually prove deleterious to the host. Some examples of immune disease and immune system-mediated injury have been extensively investigated, including anaphylactic shock, auto-immune disease, and immune complex disorders.

Recent advances in humoral and cellular immunology, molecular biology and pathology have influenced current thinking about inflammation and auto-immunity being a component of immune-mediated disease. These advances have increased our understanding of the basic aspects of antibody, B-cell, and T-cell diversity, the generation of innate (effected by monocytes, macrophages, granulocytes, natural killer cells, mast cells, γδ T-cells, complement, acute phase proteins, and such) and adaptive (T- and B-cells and antibodies) or cellular and humoral immune responses and their interdependence, the mechanisms of (self)-tolerance induction and the means by which immunological reactivity develops against self- and non-self antigenic constituents. In general, T-lymphocytes play a pivotal role in initiating the immune-mediated disease process (Sempe et al. 1991, Miyazaki et al. 1985, Harada et al. 1986, Makino et al. 1986). CD4+ T-cells can be separated into at least two major subsets, T-helper 1 and 2 (Th1 and Th2). Activated Th1 cells secrete IFN-γ and, TNF-α, while Th2 cells produce IL-4, IL-5 and IL-10. Th1 cells are critically involved in the generation of effective cellular immunity, whereas Th2 cells are instrumental in the generation of humoral and mucosal immunity and allergy, including the activation of eosinophils and mast cells and the production of IgE (Abbas et al. 1996).

In general, immune-mediated disorders are difficult to treat. Often, broad-acting medication is applied, such as treatment with corticosteroids or any other broad-acting anti-inflammatory agent that in many aspects may be detrimental to a treated individual.

Since 1900, the central dogma of immunology has been that the immune system does not normally react to self. However, it recently become apparent that auto-immune responses are not as rare as once thought and that not all auto-immune responses are harmful; some responses play a distinct role in mediating the immune response in general. For example, certain forms of auto-immune response, such as recognition of cell surface antigens encoded by the major histocompatibility complex (MHC), and of anti-idiotypic responses against self idiotypes are important, indeed essential, for the diversification and normal functioning of the intact immune system.

Apparently, an intricate system of checks and balances is maintained between various subsets of cells (i.e., T-cells) of the immune system, thereby providing the individual with an immune system capable of coping with foreign invaders. In that sense, auto-immunity plays a regulating role in the immune system.

However, it is now also recognized that an abnormal auto-immune response is sometimes a primary cause and at other times a secondary contributor to many human and animal diseases. Clinical and laboratory data of various types of auto-immune disease frequently overlap with each other and with other inflammatory disorders (e.g., transplant rejection, Alzheimer disease or due to trauma, bums or bacterial, viral or parasitic infection). More than one auto-immune and/or other inflammatory disorder tends to occur in the same individual, especially in those with auto-immune endocrinopathies. Auto-immune syndromes may be mediated with lymphoid hyperplasia, malignant lymphocytic or plasma cell proliferation and immunodeficiency disorders such as hypogammaglobulinaemia, selective Ig deficiencies and complement component deficiencies.

Examples of auto-immune diseases are: systemic lupus erythematosus (SLE), rheumatoid arthritis (RA), post-partum thyroid dysfunction, auto-immune thromocytopenia, psoriasis, scleroderma, dermatitis herpetiformis, polymyositis, dermatomyositis, pemphigus vulgaris, spondyloarthropathies such as vitiligo, ankylosing spondylitis, Sjögren's syndrome, multiple sclerosis (MS), Crohn's disease, myasthenia gravis, ulcerative colitis, auto-immune neuropathies, primary biliary cirrhosis such as Guillain-Barré, auto-immune hepatitis, auto-immune uveitis, Type 1 or immune-mediated diabetes mellitus (DM1), auto-immune hemolytic anemia, pernicious anemia, Grave's disease, auto-immune thrombocytopenia, Hashimoto's thyroiditis, auto-immune oophoritis and orchitis, auto-immune disease of the adrenal gland, anti-phospholipid syndrome, vasculitides such as Wegener's granulomatosis, Behcet's disease. In addition to these more or less “classical” examples of auto-immune disease, recent data indicate that some psychiatric or affective disorders also have an inflammatory component. Evidence supports that macrophages, as well as lymphocytes and their products, may be involved in the pathophysiology of affective disorders, major/unipolar depression, schizophrenia, seasonal affective disorder, post-partum depression, Alzheimer's disease, bipolar disorder (see Stasny et al. 2003; Pollmacher et al. 2002; Maes et al. 2001; Leu et al. 2001; Maes et al. 1995; Anisman et al. 2003; Leonard 2001). Bipolar disorder, previously known as bipolar or manic depression, is a serious, double-edged mental illness. In contrast to the sustained bleakness of generalized depression (technically described as unipolar disorder), bipolar disorder is characterized by cyclical swings between elation and despair. It has been reported that the immune system is activated in bipolar patients. For example, the T-cell system is activated in both symptomatic and euthymic patients with bipolar disorder (Breunis et al., “High numbers of circulating activated T-cells and raised levels of serum IL-2 receptor in bipolar disorder,” Biol. Psychiatry 2003, 53(2):157). An earlier study showed that thyroid auto-immunity is highly prevalent in samples of outpatients with bipolar disorder (Kupka et al., Biol. Psychiatry 2002, 15; 51(4):305).

Auto-immune diseases are characterized by auto-immune responses, for example, directed against widely distributed self-antigenic determinants, or directed against organ- or tissue-specific antigens. Such disease may follow abnormal immune responses against only one antigenic target, or against many self antigens, or even due to trauma. In many instances, it is not clear whether auto-immune responses are directed against unmodified self-antigens or self-antigens that have been modified or resemble any of numerous agents, such as viruses, bacterial antigens and haptenic groups.

There is as yet no established unifying concept to explain the origin and pathogenesis of the various auto-immune disorders. Studies in experimental animals support the notion that auto-immune diseases may result from a wide spectrum of genetic and immunological abnormalities that differ from one individual to another and may express themselves early or late in life depending on the presence or absence of many superimposed exogenous (viruses, bacteria, trauma) or endogenous (hormones, cytokines, abnormal genes) accelerating factors.

The diagnosis of chronic inflammatory and/or auto-immune disease is typically based on an individual's symptoms, findings from a physical examination and results from laboratory tests. In some cases, a specific diagnosis can be made. A diagnosis shortly after onset of a patient's symptoms will allow for early aggressive medical therapy; and for some diseases, patients will respond completely to treatments if the reason for their symptoms is discovered early in the course of their disease. If an individual has skeletal symptoms such as joint pain and a positive but non-specific lab test, she or he may be diagnosed with the confusing name of early or “undifferentiated” connective tissue disease. In this case, a physician may want the patient to return frequently for follow up. The early phase of disease may be a very frustrating time for both the patient and physician. On the other hand, symptoms may be short-lived, and inconclusive laboratory tests may amount to nothing of a serious nature. Thus, auto-immune diseases are often difficult to diagnose, particularly early in the course of the disease. Symptoms of many auto-immune diseases, such as fatigue, are non-specific. Laboratory test results may help but are often inadequate to confirm a diagnosis.

In view of the above, there is a clear need for improved and alternative methods to diagnose, screen for, or predict the development of chronic inflammatory diseases, including affective disorders such as bipolar disorder.

### DISCLOSURE OF THE INVENTION

The invention now provides the insight that a number of genes are differentially expressed in patients suffering from an inflammatory disease when compared to healthy subjects. The differential expression level of 15 inflammatory-specific genes provides a basis for new clinically applicable tools to diagnose inflammatory disease, as well as to screen for patients who are at increased risk of developing an inflammatory disease.

Provided herein is a method to diagnose or screen for an inflammatory disease in a subject, preferably a human subject, the method comprising determining the level of at least one, preferably at least two, more preferably at least three, most preferred at least four, inflammatory-specific gene product(s) in a biological sample, preferably peripheral blood monocytes isolated from the subject, wherein the inflammatory-specific gene product is selected from the group comprising HSPC228, 34703_f_a, MCP-3, CCL2, EMP1, CDC42, TLE3, SPRY2, p40BBP, HSPC060, NAB2, HSPA1A, HSPA1B, MAPRE2 and OAS1.

According to the invention, an elevated level of the HSPC228, 34703_f_at, MCP-3, CCL2, EMP1, CDC42, TLE3, SPRY2, p40BBP, HSPC060 and/or NAB2 gene product(s) and/or a reduced level of the HSPA1A, HSPA1B, MAPRE2 and/or OAS1 gene product(s) compared to the level of said gene product(s) in a sample of a healthy subject is indicative of an inflammatory disease or an increased risk of developing such a disease. Thus, a method is provided for the diagnosis or detection of an inflammatory disease in a subject by assaying for a marker of an inflammatory disease. Such a method is very useful for diagnosing and/or monitoring the development of preclinical stages of such a disease in a patient and thus presents a valuable opportunity to initiate preventive or ameliorative treatment of the disease. The term “inflammatory disease” as used herein refers to various immune-mediated disorders, including chronic inflammatory disease, auto-immune disease, and affective disorders.

HSPC228 encodes a hypothetical protein with the GenBank accession number M27826. 34703_f_at refers to zo30b03.r1 Stratagene colon (#937204). Homo sapiens cDNA clone IMAGE:588365 5′ contains LTR7.b3 LTR7 repetitive element mRNA sequence. The accession number of 34703_f_at is AA151971.

MCP-3 refers to monocyte chemotactic protein 3, also known as chemokine (C-C motif) ligand 7 (CCL7) or small inducible cytokine A7 (SCA7). The accession number of MCP-3 is X72308. CCL2 refers to human JE gene encoding a monocyte secretory protein (exon 3), also known as chemokine (C-C motif) ligand 2, monocyte chemotactic protein 1 (MCP1), small inducible cytokine A2 (SCYA2) or monocyte chemotactic and activating factor (MCAF). The GenBank accession numbers of CCL2 are M28225 and M26683. EMP1 refers to epithelial membrane protein 1, with the GenBank accession numbers Y07909 and U43916. CDC42 refers to cell division cycle 42 (GTP-binding protein, 25 kD). The accession number of CDC42 is M35543. TLE3 refers to transducin-like enhancer of split 3, homolog of Drosophila E (sp1). The GenBank accession number of TLE3 is M99438. SPRY2 refers to sprouty (Drosophila) homolog 2. The GenBank accession number of SPRY2 is AF039843. p40BBP refers to a protein that is likely to be an ortholog of rat brain-specific binding protein. The accession number of p40BBP is AL080235. HSPC060 encodes a hypothetical protein and has the GenBank accession number AF150247. NAB2 refers to NGFI-A binding protein 2 (EGR1 binding protein 2). The accession number of NAB2 is X70991. HSPA1A and HSPA1B refer to the 70 kD heat shock protein (Hsp70) 1A and 1B, respectively. They are also known as MHC class III HSP70-1 and HSP70-2. The accession numbers are M11717 (HSPA1A) and M59830 (HSPA1B). MAPRE refers to microtubule-associated protein, RP/EB family, member 2. The accession number of MAPRE is X94232. OAS1 refers to Homo sapiens 2′-5′ oligoadenylate synthetase gene, exon 8 and E18 isoform, complete cds, or to 2′,5′-oligoadenylate synthetase 1 (40-46 kD). The accession numbers of OAS1 are M11810 and X04371.

In a preferred embodiment, determining the level of an inflammatory-specific gene product comprises determining the level of the product in an isolated sample from a subject suspected of developing an inflammatory disease relative to the amount of the specific gene product in a control sample. Suitable control samples include biological samples isolated from healthy subjects. It is also possible to determine the level of an inflammatory-specific gene product in a sample relative to the level of a housekeeping gene product in the same patient sample. Generally speaking, housekeeping genes are constitutively expressed genes that serve to maintain cellular function. As such, they are presumed to produce the minimally essential transcripts necessary for normal cellular physiology. With the advent of microarray technology, it has recently become possible to identify at least a “starter set” of housekeeping genes, as exemplified by the work of Velculescu et al. (Nat. Genet. 23:387-388, 1999) who examined the expression of 7,000 full-length genes in 11 different human tissues, both adult and fetal, to determine the suite of transcripts that were commonly expressed throughout human development and in different tissues. They identified 535 transcripts via microarray hybridization as likely candidates for housekeeping genes, also referred to as “maintenance” genes. Preferably, however, a control sample comprises a defined amount of an isolated inflammatory-specific gene product, such as a vector with an inflammatory-specific gene, purified RNA of an inflammatory-specific gene, or protein encoded by an inflammatory-specific gene.

In one embodiment of the invention, the level of an inflammatory-specific gene product is determined at the DNA or RNA level, preferably at the mRNA level. As is exemplified in the Detailed Description, the expression profile of human monocytes isolated from subjects suffering from different inflammatory diseases (diabetes mellitus Type 1 and Bipolar depression) was compared to the expression profile in monocytes of healthy subjects using a U95Av2 GeneChip microarray from Affymetrix. The U95Av2 GeneChip contains over 12,600 transcripts of known genes and expressed sequence tags (ESTs). Surprisingly, it was found that the expression level of 15 human genes was specifically altered in patients with an inflammatory disease. The term “inflammatory-specific gene product” as used herein refers to a product (nucleic acid sequences as well as proteinaceous substances) that is encoded by one of these 15 genes. The expression level of four genes (HSPA1A, HSPA1B, MAPRE2 and OAS1) was reduced in patients with an inflammatory disease, whereas the expression of eleven genes (HSPC228, 34703_f_at, MCP-3, CCL2, EMP1, CDC42, TLE3, SPRY2, p40BBP, HSPC060, NAB2) was elevated in these patients. For example, the so-called heatmap in FIG. 1 shows that CCL2 is highly expressed in patients who suffer from either Type 1 diabetes mellitus (DM1) or bipolar depression (BP), when compared to age-matched DM1 or BP controls. Also, whereas HSPC228 is only expressed at a relatively low level in patients, HSPC228 expression is much lower in controls. In contrast, the expression of, for example, HSPA1A and HSPA1B are clearly reduced in both DM1 and BP patients when compared to controls.

Thus, evaluation of the expression level of at least one of these 15 inflammatory-specific genes (“inflammatory markers”) allows diagnosis or screening for an inflammatory disease because an up-regulation of HSPC228, 34703_f_at, MCP-3, CCL2, EMP1, CDC42, TLE3, SPRY2, p40BBP, HSPC060 or NAB2, or a down-regulation of HSPA1A, HSPA1B, MAPRE2 and OAS1 expression is only found in patients and not in healthy subjects (FIG. 1). Herewith, we have provided diagnostic markers for inflammatory disease, in particular, auto-inflammatory or auto-immune disease.

In a further aspect of the invention, these markers are advantageously used as prognostic markers because they also have prognostic value with regard to disease development and/or complications, e.g., after trauma or infectious disease (e.g., Guillain-Barré syndrome). To this end, appropriate cell samples of such a subject can be cultured in vitro with an appropriate stimulus, e.g., a lectin, lipopolysaccharides or stimulating antibody. Using the markers provided herein, it is possible to discriminate between different conditions on the basis of different expression patterns. A specific combination of differentially expressed genes can be indicative of an increased risk of developing a specific disease or condition, for example, in response to trauma or a bacterial, viral or parasitic infection. Furthermore, determination of one or more genes with a dysregulated expression level can be used to determine the stage or severity of an inflammatory disorder or disease.

Next, using the same GeneChip approach as described above, genes were identified that were specifically up- or down-regulated in patients with a major affective disorder (in this case, patients with bipolar disorder (BP) receiving lithium therapy), but not in. DM1 patients or age-matched controls. As is depicted in FIG. 2, the expression level of CCR2, CX3CR1 and DOK1 is reduced in BP patients when compared to age-matched controls, whereas an increased expression was observed for HBB, G-gamma globulin, THBD, PHLDA1, DTR and GNLY. CCR2 refers to the chemokine (C-C motif) receptor 2. The GenBank accession numbers of CCR2 are U03905, U95626 and U03905. CX3CR1 refers to the chemokine (C-X3-C) receptor 1, with the accession number U20350. DOK1 refers to the 62 kD docking protein 1 (reported to act downstream of tyrosine kinase 1). The accession numbers of DOK1 are AF035299 and U70987. HBB refers to hemoglobin beta. HBB expression in BP patients was approximately 60-fold increased when compared to healthy controls. The accession number of HBB is M25079. G-gamma globulin refers to Homo sapiens G-gamma globulin (G-gamma globin) and A-gamma globin genes, complete cds. The accession number of G-gamma globulin is M91036. THBD refers to human thrombomodulin gene, complete cds. The accession number of THBD is J02973. PHLDA1 refers to pleckstrin homology-like domain, family A, member 1. The accession number of THBD is J02973. DTR refers to diphtheria toxin receptor (heparin-binding epidermal growth factor-like growth factor). The accession number of DTR is M60278. GNLY refers to granulysin, with the accession number M85276.

Herewith, the invention provides a method to diagnose or screen for affective disorder (AD) in a subject, preferably a human subject, the method comprising determining the level of at least one, preferably at least two, more preferably at least three, most preferred at least four, -AD-specific gene product(s) in a biological sample isolated from the subject, preferably peripheral blood monocytes, wherein the gene is selected from the group comprising CCR2, CX3CR1, DOK1, HBB, G-gamma globin, THBD, PHLDA1, DTR and GNLY. In one embodiment, detection is performed at the mRNA level. Preferably, the increase in the HBB, G-gamma globin, THBD, PHLDA1, DTR and/or GNLY mRNA level is at least two-fold compared to the level of the mRNA in a sample of a healthy subject to be indicative of AD. Likewise, the decrease in the CCR2, CX3CR1 and/or DOK1 mRNA level is at least two-fold compared to the level of the mRNA in a sample isolated from a healthy subject. More preferred, the altered expression level of either one of these AD-specific genes according to the invention is more than three-fold, or more than four-fold, or even higher than that, when compared to controls.

Detection of an inflammatory-specific or affective disorder-specific nucleic acid sequence can be achieved by conventional techniques well known in the art. These include PCR techniques for detecting specific DNA sequences and reverse transcriptase (RT) PCR techniques for detecting specific RNA sequences. In one embodiment, a specific gene product is detected using a nucleic acid amplification assay, preferably a PCR assay, using a set of nucleic amplification primers capable of specifically amplifying the gene product. More preferred, the PCR assay is a real-time quantitative PCR (RQ-PCR) assay. The simplest RQ-PCR technique is based on detection of PCR products by the DNA-intercalating dye SYBR Green I. Other suitable RQ-PCR analyses involve those using fluorochrome-labeled sequence-specific probes including hydrolysis probes or hybridization probes.

In a preferred embodiment of the invention, the increase in the HSPC228, 34703_f_at, MCP-3, CCL2, EMP1, CDC42, TLE3, SPRY2, p40BBP, HSPC060, NAB2, CCL2, MCP-3, EMP1, STX1A, CD9, PTPN7, CDC42, FABP5 and/or NAB2 mRNA level is at least two-fold compared to the level of mRNA in a sample of a healthy subject to be indicative of an inflammatory condition or disease. Likewise, the decrease in the HSPA1A, HSPA1B, MAPRE2 and/or OAS1 mRNA level is at least two-fold compared to the level of mRNA in a sample of a healthy subject. More preferred, the altered expression either one of these inflammatory-specific genes is more than three-fold, or even more than four-fold, or even higher than that, when compared to control.

As said, the BP-specific gene products identified above were obtained using BP subjects who received lithium therapy. Numerous in vivo effects of lithium therapy have been reported, including the observation that chronic lithium regulates transcriptional factors, which in turn may modulate the expression of a variety of genes that compensate for aberrant signaling associated with the pathophysiology of bipolar disorder (see, for a review: R. H. Lenox and C. G. Hahn, Overview of the mechanism of action of lithium in the brain: fifty-year update, J. Clin. Psychiatry, 2000; 61 Suppl. 9:5-15). Therefore, we also investigated the gene expression pattern using U95Av2 GeneChips in patients with BP who did not receive lithium therapy (see Example 2). Furthermore, children of BP parents were analyzed who developed BP during the course of the study to identify a set of “pre-BP-specific” gene products. Data analysis was performed by Affymetrix GeneChip Operating Software 1.1. The results are summarized in Table 1. Therefore, the invention relates to a method to diagnose, screen for or predict the development of an affective disorder (AD), preferably bipolar disorder (BP), in a subject, the method comprising determining the level of at least one, preferably at least two, more preferably at least three, most preferred at least four, AD-specific gene product(s) in a biological sample isolated from the subject, preferably peripheral blood monocytes, wherein the gene is selected from the group comprising ATF3, phosphodiesterase 4 B, CXCL2, BCL2-related protein A2, dual specificity phosphatase 2, TNFα-induced protein 3/A20, BTEB1, CXCL3, Chemokine CCL-3 like, CCL-4, CCL20, CX2CR1, amphiregulin, thrombomodulin, heparin-binding EGF-like growth factor, DNA-damaged inducible transcript, V28 chemokine-like receptor, TRAIL, MAPK6, E4BP4, PBEF1, thrombospondin 1, MAFF, HSP70, CCL2, MCP-3, CCR2, CX3CR1, DOK1, HBB, G-gamma globin, THBD, PHLDA1, DTR and GNLY.

The pre-bipolar gene expression pattern is characterized by a significant reduction (compared to age- and sex-matched controls) in the expression level of ATF3 (L19871), Phosphodiesterase 4 B (L20971), CXCL2 (MIP2) (M36820), BCL2-related protein A2/Bfl-1 (U27467), Dual specificity phosphatase 2/DUSP2/PAC1 (L11329), TNFα-induced protein 3/A20 (M59465), BTEB1 (transcription factor) (D31716), CXCL3 (M36821), Chemokine CCL-3 like (D90144), CCL-4 (J04130), CCL20 (U64197), TNF (X02910), IL-1β, IL-1β (M15330), IL-6 (X04430), TNF (M16441) and CX2CR1 (M20350). The pre-bipolar samples showed a significantly increased expression level of Amphiregulin (M30704), CD54 (ICAM1) (M24283), Thrombomodulin (J02973), Heparin-binding EGF-like growth factor (M60278), DNA-damaged inducible transcript (DDIT4) (AA522530), V28 chemokine-like receptor (MCP-1, MIP-1 (U20350) and TNFα super family, member 10/TRAIL (U37518). Thus, these gene products are advantageously used to predict the development of an affective disorder, in particular BP.

Bipolar patients not receiving lithium therapy showed a significant increase in the expression of ATF3, Phosphodiesterase 4 B, CXCL2, BCL2-related protein A2/Bfl-1, Dual specificity phosphatase 2/DUSP2/PAC1, TNFα-induced protein 3/A20, MAPK6 (X80692), E4BP4 (X64318), PBEF1 (U02020), Thrombospondin 1 (X14787) and MAFF (AL021977). Small yet probably insignificant increases were observed for BTEB1 (transcription factor), CXCL3, Chemokine CCL-3 like, CCL-4, CCL20, TNF, IL-1β, IL-1β (M15330), IL-6 and TNF. These BP-specific gene products are advantageously used to diagnose or screen for an affective disorder, in particular BP.

Analysis of bipolar subjects receiving lithium therapy showed increased expression of ATF3, Amphiregulin, CD54 (ICAM1), Thrombomodulin, Heparin-binding EGF-like growth factor, DNA-damaged inducible transcript (DDIT4), V28 chemokine-like receptor (MCP-1, MIP-1), E4BP4, PBEF1, Thrombospondin 1, CCL2 (M28225), CCL2 (M26683) and MCP3 (X72308). A reduced expression level was observed in lithium-treated BP subject for CX2CR1, TNF super family, member 10/TRAIL (U37518), MAPK6 and HSP70 (M59830).

A method of the invention is easily practiced in routine laboratory settings. Specific gene products according to the invention can be analyzed in various types of cells that are easily obtained from a subject, including monocytes, dendritic cells, T-cells, granulocytes, natural killer T-cells and other (natural) killer cells. All that is required is obtaining or isolating a sample from the subject, preferably a peripheral blood sample, isolating the cell type of interest (preferably monocytes), optionally preparing an extract of the sample (e.g., nucleic acid extract or a total cell lysate) and determining the level of a specific gene product in the sample or in an extract thereof. The level can be determined by contacting a cellular component of the sample with at least one reagent specifically reactive with an inflammatory-specific gene product and, optionally, with at least one reagent specifically reactive with a housekeeping gene product. The reagent or reagent(s) may be labeled and they may be immobilized on a solid support, for example, a glass, nylon or nitrocellulose solid support. Also provided are reagents, such as nucleotide probes and antibodies specifically reactive with an inflammatory-specific gene product or with a fragment thereof.

In another embodiment, a nucleic acid extract of a sample isolated from a subject is contacted with at least one nucleotide probe comprising a sequence that hybridizes to a nucleotide region encoding an inflammatory- or BP-specific gene and, optionally, with at least one nucleotide probe comprising a sequence that hybridizes to a nucleotide region encoding a housekeeping gene. At least one nucleotide probe may be immobilized on a solid support, for example, in an array format. Examples of suitable nucleotide probes comprise DNA, RNA or cDNA probes. The nucleic acid extract may contain nucleic acids with a detectable label, e.g., biotinylated cRNA.

Different types of DNA microarrays for use in a method of the invention can be prepared according to methods known in the art, e.g., arrays based on standard microscopic glass slides on which cDNAs or long oligonucleotides (typically 70-80 mers) have been spotted. Another type is based on photolithographic techniques to synthesize 25-mer oligonucleotides on a silicon wafer and constitutes the patented Affymetrix technology.

In a further embodiment, a method to diagnose or screen for an auto-inflammatory condition or disease or affective disorder in a subject comprises determining the level of at least one inflammatory or specific gene product at the protein level. To this end, an extract is prepared from a sample isolated from a subject that allows the specific detection of a protein, for instance, by preparing a total cell lysate in the presence of a chaotropic agent such as a detergent or a salt. A sample (comprising at least one cell) from the subject may be pretreated prior to preparing an extract to improve detection of an inflammatory-specific gene product. For example, some proteins including CCL2 and MCP-3 are known to be rapidly secreted following their synthesis. Pretreatment with a protein secretion inhibitor (e.g., monensin) can thus be used to accumulate a specific protein within the cell cytoplasm and therewith increase the content of one or more inflammatory-specific gene products in an extract. An extract of a sample can subsequently be contacted with a specific binding partner of at least one protein (or fragment thereof) encoded by a specific gene product under conditions that allow formation of a complex between the binding partner and the protein and detecting the complex formation. The amount of complex formed is indicative of the amount of the inflammatory-specific protein present in the sample extract. Therefore, it is advantageous to use a binding partner that is provided with a detectable label, such as a fluorochrome, radioactive label, enzyme, etc. A well-known method known in the art to specifically detect a protein involves the immunological detection of a protein using a specific monoclonal or polyclonal antibody or fragment thereof (e.g., single chain Fv) as a specific binding partner.

Monoclonal antibodies may be obtained by well-established methods, e.g., as described in A. Johnstone and R. Thorpe, Immunochemistry in Practice, 2nd Ed., Blackwell Scientific Publications, 1987, pp. 35-43. When prepared by recombinant DNA techniques, the antibody may be produced by cloning a DNA sequence coding for the antibody or a fragment thereof into a suitable cell, e.g., a microbial, plant, animal or human cell, and culturing the cell under conditions conducive to the production of the antibody or fragment in question and recovering the antibody or fragment thereof from the culture. Possible strategies for the preparation of cloned antibodies are discussed in, for instance, L. Riechmann et al., Nature 332, Mar. 24, 1988, p. 323 ff., describing the preparation of chimeric antibodies of rat variable regions and human constant regions; M. Better et al., Science 240, May 20, 1988, p. 1041 ff., describing the preparation of chimeric mouse-human Fab fragments; A. Sharra and. A. Pluckthun, Science 240, May 20, 1988, pp. 1038-1040, describing the cloning of an immunoglobulin Fv fragment containing antigen-binding variable domains; and E. S. Ward et al., Nature 341, Oct. 12, 1989, pp. 544-546, describing the cloning of isolated antigen-binding variable domains (“single-domain antibodies”).

In one aspect of the invention, a method is provided to diagnose or screen for, or predict the development of an inflammatory condition or disease in a subject, wherein the method comprises the immunological detection of at least one, preferably at least two, more preferably at least three inflammatory-specific protein(s) in a biological sample isolated from the subject, wherein the protein is encoded by a gene selected from the group comprising HSPC228, 34703_f_at, MCP-3, CCL2, EMP1, CDC42, TLE3, SPRY2, p40BBP, HSPC060, NAB2, HSPA1A, HSPA1B, MAPBE2 and OAS1. In a second aspect, a method is provided to diagnose or screen for, or to predict the development of an affective disorder in a subject wherein the method comprises the immunological detection of at least one, preferably at least two, more preferably at least three AD-specific protein(s) in a biological sample isolated from the subject, wherein the protein is encoded by a gene selected from the group comprising CCR2, CX3CR1, DOK1, HBB, G-gamma globin, THBD, PHLDA1, DTR and GNLY.

Immunological detection is a common tool used in discovering protein expression patterns, isolating proteins from cellular extracts and in screening cells and extracts for specific proteins. The immunological detection in a method of the invention can be performed according to well-known assays, including Western blotting, immunoprecipitation assays, enzyme-linked immunosorbant assays (ELISA), dot-blot assays and chip-based assays. Detailed practical information regarding the immunological detection of proteins can be found in “Antibodies: A Laboratory Manual” by Ed Harlow and David Lane (Editors) ISBN: 0879695447, Publisher: Cold Spring Harbor. In one embodiment, a method is provided for diagnosing or predicting BP comprising detecting ATF3 at the protein level. ATF3 (Activating transcription factor 3) is a member of the ATF/CREB family of transcription factors and its expression is increased by various pathophysiological conditions and in several cancer cells. Anti-ATF3 antibody is commercially available from Santa Cruz Biotechnology.

In another aspect of the invention, an inflammatory or affective disorder ELISA test is provided that is based on the principle of a solid phase enzyme-linked immunosorbant assay. As an example, a CCL2-ELISA test is described. The assay system utilizes a monoclonal antibody directed against an antigenic determinant of CCL2 that is used for solid phase immobilization (e.g., in microtiter wells). Monoclonal antibodies against human CCL-2 are commercially available, for example, from Alexis Biochemicals (Alexis Benelux, Breda, The Netherlands). A goat antibody conjugated to horseradish peroxidase (HRP) reactive with a different antigenic determinant of CCL2 is present in an antibody-enzyme conjugate solution. The test sample (e.g., cell lysate) is allowed to react simultaneously with the two antibodies, resulting in the CCL2 molecule being sandwiched between the solid phase and enzyme-linked antibodies. After a one-hour incubation at room temperature, the wells are washed with water to remove unbound labeled antibodies. A solution of HRP-substrate, for instance, 3,3′,5,5′-tetramethylbenzidine (TMB), is added and incubated for 20 minutes, resulting in the development of a blue color. The color development is stopped with the addition of a stop solution (e.g., 1 N HCl) changing the color to yellow. Absorbance is measured spectrophotometrically at 450 nm. The concentration of CCL2 is directly proportional to the ;color intensity of the test sample. An increase in the concentration of CCL2 in a sample is indicative of an inflammatory condition or disease. Of course, it is preferred to include a standard curve of known amounts of the CCL2 protein in order to quantitate the amount of CCL2 present. Furthermore, the detection of a protein that is not subject to a change in patients with an auto-inflammatory disease, for example, a housekeeping enzyme, may be used as internal control for the amount of protein assayed and/or as a control between different samples. In a similar fashion, an AD-ELISA test can be designed.

In a further embodiment, a test kit is provided for diagnosing or screening for an inflammatory disease, such as Diabetes Mellitus Type 1 (DM1) or affective disorder, in a subject comprising at least one reagent specifically reactive with a gene product selected from the group comprising HSPC228, 34703_f_at, MCP-3, CCL2, EMP1, CDC42, TLE3, SPRY2, p40BBP, HSPC060, NAB2, HSPA1A, HSPA1B, MAPRE2 and OAS1. The kit optionally further comprises at least one reagent specifically reactive with a housekeeping gene product. A suitable reagent can be an antibody or fragment thereof or a nucleotide probe specifically reactive with an inflammatory-specific gene product. The reagent may be immobilized on a solid support, preferably a glass, nylon or nitrocellulose solid support. The physical shape of the solid support is not critical, although some shapes may be more convenient than others for the present purpose. Thus, the solid support may be in the shape of a plate, e.g., a microtiter plate or a paper strip, dipstick, membrane (e.g., a nylon membrane or a cellulose filter) or solid particles (e.g., latex beads). Furthermore, a kit preferably also comprises a defined amount of one or more inflammatory-specific gene products that can serve as a control sample. A vector with such a specific gene, (purified) RNA of a specific gene, or (purified) protein encoded by an inflammatory-specific gene can be used as a quantitative control.

For example, a test kit is provided comprising an array of nucleotide probes comprising at least ten nucleotide bases in length, wherein at least one probe hybridizes to a fragment of at least one inflammatory-specific gene selected from the group comprising HSPC228, 34703_f_at, MCP-3, CCL2, EMP1, CDC42, TLE3, SPRY2, p40BBP, HSPC060, NAB2, HSPA1A, HSPA1B, MAPRE2 and OAS1, and optionally, wherein at least one probe hybridizes to a fragment of at least one housekeeping gene. Of course, the different probes should be specific for the different inflammatory-specific genes. The array of nucleotide probes is, for instance, arranged on a solid support in multiple discrete regions of distinct nucleic acid strands. In another example, a kit according to the invention to diagnose or screen for an inflammatory disorder or disease comprises one or more antibodies specifically reactive with an inflammatory-specific protein. The antibodies can be labeled to aid in detection of an antibody-antigen complex. The label substance for the reagents is preferably selected from the group consisting of enzymes, colored or fluorescent substances and radioactive isotopes.

Examples of enzymes useful as label substances are peroxidases (such as horseradish peroxidase), phosphatases (such as acid or alkaline phosphatase), β-galactosidase, urease, glucose oxidase, carbonic anhydrase, acetylcholinesterase, glucoamylase, lysozyme, malate dehydrogenase, glucose-6-phosphate dehydrogenase, β-glucosidase, proteases, pyruvate decarboxylase, esterases, and luciferase, etc. Enzymes are not in themselves detectable but must be combined with a substrate to catalyze a reaction, the end product of which is detectable. Thus, a substrate may be added after contacting the support with the labeled reagent, resulting in the formation of a colored or fluorescent substance. Examples of substrates that may be employed according to the invention include hydrogen peroxide/tetramethylbenzidine or chloronaphthole or o-phenylenediamine or 3-(p-hydroxyphenyl) propionic acid or luminol, indoxyl phosphate, p-nitrophenylphosphate, nitrophenyl galactose, 4-methyl umbelliferyl-D-galactopyranoside, or luciferin. Alternatively, the label substance may comprise colored or fluorescent substances, including Europium, gold particles, colored or fluorescent latex particles, dye particles, fluorescein, phycoerythrin or phycocyanin. Radioactive isotopes that may be used for the present purpose may be selected from I-125, I-131, H-3, P-32, P-33 and C-14.

Preferably, a kit further comprises standard amounts of (lyophilized) inflammatory-specific protein and, optionally, other reagents required for immunological detection of a protein such as a binding buffer, enzyme substrate, stop solution, and the like. An inflammatory-specific protein may be obtained using recombinant expression of a nucleic add sequence encoding the inflammatory-specific protein in a host cell, preferably followed by purification of the protein. Of course, in a similar fashion, using AD-specific genes, test kits and arrays can be designed to diagnose, screen for or to predict affective disorder.

A method of the invention, a kit as defined above or an array of probes is advantageously used in the diagnosis or screening for increased risk of developing an auto-inflammatory disease or an affective disorder in a subject, preferably a human subject.

### BRIEF DESCRIPTION OF THE FIGURES AND TABLE

FIG. 1: Microarray analysis of DM1 patients, PB patients (receiving lithium therapy), DM2 patients and their sex- and age-matched controls. Purified monocytes were pooled and analyzed for gene expression using DNA microarrays. The relative expression levels of the selected inflammatory-specific genes across DM1 patients, DM1 controls, BP patients and BP controls are shown in a heatmap. Intensities were normalized across all intensity experiments and expressed on a gray scale by use of the software Rosetta Resolver. The increase (white) and decrease (black) in expression level relative to the median value are shown for the six experiments. The genes were also clustered using agglomerative clustering algorithm. Rows represent the relative intensity in the six groups. Columns represent the genes. Each cell in the heatmap represents a Z-score.

FIG. 2: Scatter plot of intensity of genes in BP patients receiving lithium therapy (Y-axis) versus sex- and age-matched Bp controls (X-axis) is shown. Expression levels were averaged (weighted on basis of error) across two replicate experiments.

Table 1. Results of the GeneChip analysis of pre-bipolar subjects and bipolar subjects not receiving lithium therapy, performed as described in Example 2. Arrows ↑ and ↓ indicate, respectively, a significant increase or decrease in expression level compared to age- and sex-matched controls, whereas (n.s.) indicates a non-significant change. ˜means that no change was observed. The GenBank accession number of each gene product is indicated between brackets. The last column shows the results obtained in parallel for bipolar subjects receiving lithium therapy (see Example 1 for details).

### DETAILED DESCRIPTION OF THE INVENTION

### EXAMPLES

### Example 1

Identification of Inflammatory Disease Markers

Experimental Procedures

Subjects

Peripheral, blood mononuclear cells (PBMCs) were isolated from heparinized peripheral blood using Ficoll-Paque (Amersham Biosciences, Uppsala, Sweden) density gradient centrifugation, resuspended in RPMI1640 medium containing HEPES and ultraglutamin supplemented with 10% fetal calf serum and 10% DMSO, and stored at −180° C. providing a bank for experiments. This study was approved by the Medical Ethical Committee of the Erasmus MC, Rotterdam, The Netherlands. All subjects provided written informed consent prior to participation.

At the start of an experiment, the PBMCs were quickly thawed and pooled into patient samples or control samples to minimize individual variations. Seven Bipolar Disorder (BP) patients were pooled into BP samples (two different pools; mean age 42.1 years), seven healthy controls for BP patients (CoBP; sex- and age-matched) into CoBP samples (two different pools). The BP patients were on lithium therapy. Eight to nine recent onset type 1 Diabetes Mellitus (DM1) patients were pooled into DM1 samples (three different pools; mean age 14.4 years), seven to eight healthy controls for DM1 patients (CoDM1; age-matched) into CoDM1 samples (three different pools).

The pooled cell samples were labeled with anti-CD14 magnetic beads (Miltenyi Biotec GmbH, Bergisch Gladbach, Germany) for 20 minutes at 4° C. and separated into CD14+ and CD14− fractions using the autoMACS (Miltenyi Biotec), program positive selection sensitive. The CD 14+ fraction represents the monocytes with a purity of at least 94%.

RNA Isolation

Total RNA was extracted from the monocytes with the use of an RNeasy Mini kit and QIAshredders from Qiagen (Westburg, Leusden, The Netherlands) according to the manufacturer's instructions. The integrity of the RNA was tested on 1.2% formaldehyde containing agarose gels.

Target Preparation

DNA complementary to the total RNA samples was synthesized from 4-5 μg of total RNA using a Superscript Double-Stranded Synthesis kit from Invitrogen (Breda, The Netherlands) and a T7-(dT)24 primer (GenSet Oligos, Paris, France) according to the manufacturer's instructions. The cDNA sewed as a template for in vitro transcription reaction (37° C. for five hours) using T7 RNA polymerase and biotinylated ribonucleotides employing an Enzo BioArray High Yield RNA transcript labeling kit (Enzo Life Sciences, Farmingdale, N.Y., USA). The cDNA and cRNA was purified using the GeneChip Sample Cleanup module (Affymetrix, Santa Clara, Calif., USA). The cRNA was quantified by spectrophotometric methods. An adjusted cRNA yield was calculated to reflect carryover of unlabeled total RNA.

Hybridization and Staining

Twenty micrograms of biotinylated cRNA was randomly fragmented by heat and alkaline treatment. To check the quality of the procedure, 5 μg cRNA was hybridized to a Test3 microarray (Affymetrix). Subsequently, 10 μg of cRNA was hybridized to a U95Av2 microarray (Affymetrix; HG-U95Av2 GeneChip) for 16 hours at 45° C. The U95Av2 GeneChip contains 12,600 transcripts of known genes and expressed sequence tags (ESTs) that are characterized on their function or disease association. After washing and staining with PE-conjugated streptavidin, the microarrays were scanned in an HP/Affymetrix scanner at 570 nm using a krypton/argon laser.

Data Analysis

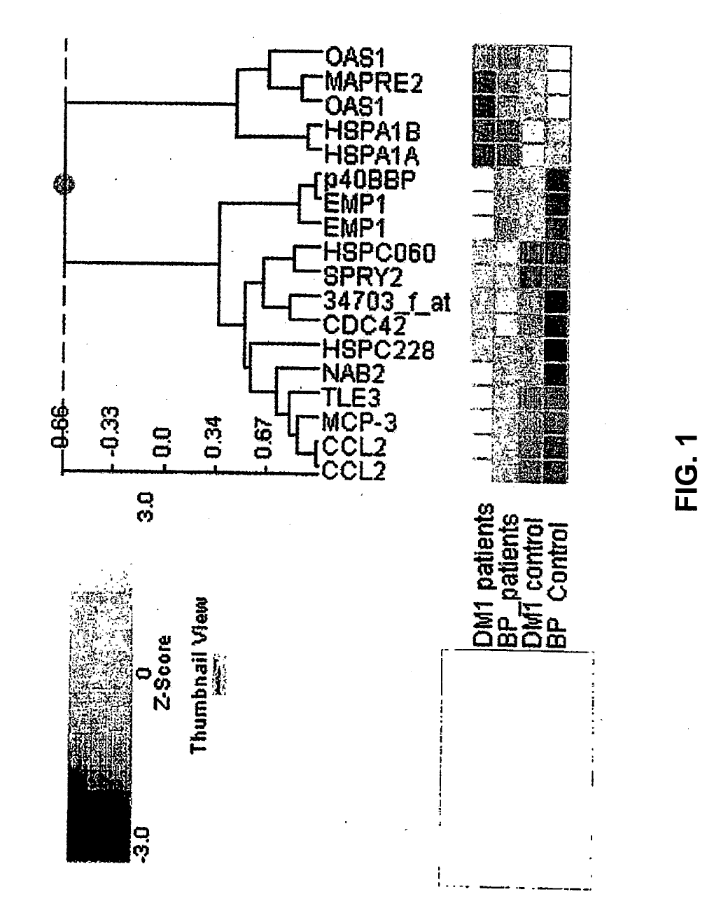

Image analysis was performed with Microarray Suite, version 5.0 (Affymetrix) using the control sample as baseline. To facilitate comparison between samples and experiments, the trimmed mean signal of each microarray was scaled to a target intensity of 2500. The expression profiles of DM1 and BP were compared to expression profiles of the CoDM1 and CoBP, respectively. The expression profiles were exported to the Rosetta Resolver Expression Data Analysis System (Rosetta Inpharmatics, Kirkland, Wash., USA) for further analyses including comparison analyses and cluster analyses. To increase confidence, the three DM1 profiles, three CoDM1 profiles, two BP profiles, and two CoBP profiles were combined in an error weighted fashion into a single DM1 experiment, a single CoDM1 experiment, a single BP experiment, and a single CoBP experiment, respectively. The data of these experiments were used to create the intensity-based ratio experiments DM1 versus CoDM1 and BP versus CoBP. The genes that were changed at least four-fold between all patients and their controls and had a p≦0.01 in these ratio experiments were grouped together as biosets (a DM1 bioset and a BP bioset). We then clustered data of the DM1, CoDM1, BP, and CoBP, experiments restricted to the genes present in both the biosets employing the agglomerative clustering algorithm. The results of this 2D cluster was displayed in a heatmap in which each cell represents a log (ratio). This heatmap was used to select inflammatory-specific genes, that is, genes that were up- or down-regulated in DM1 and BP patients, but not in CoDM1 nor in CoBP patients (see FIG. 1).

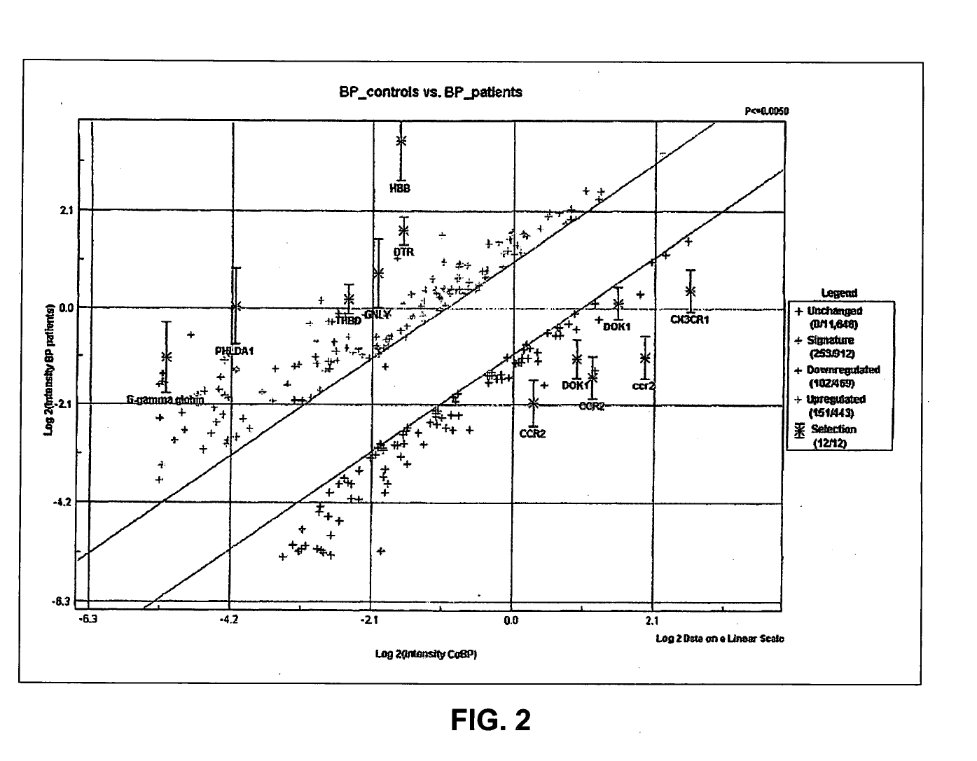

To identify BP-specific genes, the genes present in the BP bioset but not present in the DM1 bioset were further analyzed. The genes that 1) were changed at least four-fold between BP patients and their controls; 2) had a clear detectable signal (Rosetta recalculated intensity of at least 1.0) in the BP patients or in the CoBP; and 3) had a p≦0.01 in the BP versus CoBP ratio experiment, were grouped together as a new bioset (BP-specific bioset). We then plotted the intensity of the selected genes (of BP-specific bioset) in the BP patients against the intensity of those genes in BP controls (see FIG. 2).

### Example 2

Identification of BP Marker Genes

In this example, genes were identified that were specifically up- or down-regulated in both adolescent and juvenile patients with bipolar disorder (BP) who did not receive lithium therapy. The BP subjects investigated and their age- and sex-matched controls were as follows:

BP female 31 years versus control female 31 years; BP male 36.4 years versus control male 39 years. In addition, four children from parents with BP were investigated. At the onset of the study, these children did not display symptoms of BP. However, during the course of the study, they all developed BP. Thus, the genetic data obtained from these children at the beginning of the study can be regarded as “pre-bipolar.” The BP children and their age- and sex-matched controls were as follows: BP boy 11.7 years versus control boy 16 years; BP girl 12.2 years versus control boy 12 years; BP boy 16.1 years versus control boy 16 years and BP boy 20 years versus control boy 22 years. For comparison, adolescent BP subjects described in Example 1 (i.e., receiving conventional lithium therapy) were analyzed in parallel.

RNA isolation, target preparation and hybridization/staining were performed as described in Example 1.

Data Analysis

Image analysis was performed by Affymetrix GeneChip Operating Software 1.1. Background was then removed from the resulting probe intensities by robust multi-chip analysis (RMA), i.e., fitting a normal density (background) to left-of-mode mismatch (MM) probe intensity data and an exponential density (signal) to right-of-mode perfect match (PM) probe intensity data. For each probe, the background-adjusted signal was then defined as the expected signal, given the background estimate.

Next, a number of comparisons wer performed. All arrays included in a comparison were normalized to each other using quantile normalization on the perfect match (PM) probe intensities, i.e., matching their distributions exactly. Probe intensities were then log2-transformed to justify the use of an additive noise model. Next, per probeset, average expression over groups of arrays and a p-value for the statistical significance of the difference between these groups was calculated. This was done on PM probe intensities only, using a two-way analysis of variance (ANOVA) with factors “probe” and “group.” The resulting p-values were then \check{S}idák step-up adjusted to account for multiple testing.

Probesets with both a p-value<0.01 and a fold change of at least two were considered significantly differentially expressed. Sets of significant probe sets were found for children (C), adults (A) and adults who had been prescribed lithium (L). Next, intersections were taken between these sets to anus at small sets of markers. The data are summarized in Table 1.

### REFERENCES

Abbas A. K., K. M . Murphy, and A. Sher. Nature, Oct. 31 1996; 383(6603):787-93. “Functional diversity of helper T lymphocytes.”
Anisman H. and Z. Merali. Ann. Med., 2003; 35(1):2-11. “Plasma-soluble interleukin-2 and transferrin receptor in schizophrenia and major depression. Cytokines, stress and depressive illness: brain-immune interactions.”
Leonard B. E. Int. J Dev. Neurosci., June 2001; 19(3):305-12. “Changes in the immune system in depression and dementia: causal or coincidental effects?”
Leu S. J., I. S. Shiah, L. N. Yatham, Y. M. Cheu, and R. W. Lam. J. Affect. Disord., March 2001; 63(1-3):27-34. “Effects of pregnancy and delivery on the availability of plasma tryptophan to the brain: relationships to delivery-induced immune activation and early post-partum anxiety and depression.”
Maes M., H. Y. Meltzer, P. Buckley, and E. Bosmans. Eur. Arch. Psychiatry Clin. Neurosci., 1995; 244(6):325-9. “Immune-inflammatory markers in patients with seasonal affective disorder: effects of light therapy.”
Maes M., W. Ombelet, R. Verkerk, E. Bosmans, and S. Scharpe. Psychol. Med., July 2001; 31(5):847-58. Brain Behav. Immun., October 2002; 16(5):525-32. “Low levels of circulating inflammatory cytokines--do they affect human brain functions?”
Pollmacher T., M. Haack, A. Schuld, A. Reichenberg, R. Yirmiya. Brain Behav. Immun., October 2002; 16(5):525-32. “Low levels of circulating inflammatory cytokines—do they affect human brain functions?”

Stastny J., A. Konstantinidis, M. J. Schwarz, N. E. Rosenthal, O. Vitouch, S. Kasper, and A. Neumeister. Biol. Psychiatry, Feb. 15, 2003; 53(4):332-7. “Effects of tryptophan depletion and catecholamine depletion on immune parameters in patients with seasonal affective disorder in remission with light therapy.”

TABLE 1

| | | | Bipolar | Bipolar |
|---|---|---|---|---|
| | | Pre- | (no | (lithium- |
| Gene Product | | bipolar | lithium) | treated) |
| 1. | ATF3 (L19871) | ↓ | ↑ | ↑ |
| 2. | Phosphodiesterase 4 B (L20971) | ↓ | ↑ | ˜ |
| 3. | CXCL2 (MIP2) (M36820) | ↓ | ↑ | ˜ |
| 4. | BCL2-related protein A2/Bfl-1 | ↓ | ↑ | ˜ |
| | (U27467) | | | |
| 5. | Dual specificity phosphatase | ↓ | ↑ | ˜ |
| | 2/DUSP2/PAC1 (L11329) | | | |
| 6. | TNFα-induced protein 3/A20 | ↓ | ↑ | ˜ |
| | (M59465) | | | |
| 7. | BTEB1 (transcription factor) | ↓ | (n.s.) | (n.s.) |
| | (D31716) | | | |
| 8. | CXCL3 (M36821) | ↓ | (n.s.) | (n.s.) |
| 9. | Chemokine CCL-3 like (D90144) | ↓ | (n.s.) | ˜ |
| 10. | CCL-4 (J04130) | ↓ | (n.s.) | ˜ |
| 11. | CCL20 (U64197) | ↓ | (n.s.) | ˜ |
| 12. | TNF (X02910) | ↓ | (n.s.) | ˜ |
| 13. | IL-1β | ↓ | (n.s.) | ˜ |
| 14. | IL-1β (M15330) | ↓ | (n.s.) | ˜ |
| 15. | IL-6 (X04430) | ↓ | (n.s.) | ˜ |
| 16. | TNF (M16441) | ↓ | (n.s.) | ↓ (n.s.) |
| 17. | CX2CR1 (M20350) | ↓ | ˜ | ↓ |
| 18. | Amphiregulin (M30704) | ↑ | ˜ | ↑ |
| 19. | CD54 (ICAM1) (M24283) | ↑ | ˜ | ↑ |
| 20. | Thrombomodulin (J02973) | ↑ | ˜ | ↑ |
| 21. | Heparin-binding EGF-like growth | ↑ | ˜ | ↑ |
| | factor (M60278) | | | |
| 22. | DNA-damaged inducible transcript | ↑ | ˜ | ↑ |
| | (DDIT4) (AA522530) | | | |
| 23. | V28 chemokine-like receptor | ↑ | ˜ | ↑ |
| | (MCP-1, MIP-1 (U20350) | | | |
| 24. | TNF super family, member | ↑ | ˜ | ↓ |
| | 10/TRAIL (U37518) | | | |
| 25. | MAPK6 (X80692) | ˜ | ↑ | ↓ |
| 26. | E4BP4 (X64318) | ˜ | ↑ | ↑ |
| 27. | PBEF1 (U02020) | ˜ | ↑ | ↑ |
| 28. | Thrombospondin 1 (X14787) | ˜ | ↑ | ↑ |
| 29. | MAFF (AL021977) | ˜(↑) (n.s.) | ↑ | ˜(↑) (n.s.) |
| A. | HSP70 (M59830) | ↓(n.s.) | ↓(n.s.) | ↓ |
| B. | CCL2 (M28225) | ↑(n.s.) | ˜ | ↑ |
| C. | CCL2 (M26683) | ↑(n.s.) | ˜ | ↑ |
| D. | MCP3 (X72308) | ↑(n.s.) | ˜ | ↑ |

1. A method of diagnosing, screening, or predicting development of an affective disorder (AD) in a subject, said method comprising:
determining the level of from at least one to at least four AD-specific gene products in a biological sample isolated from the subject wherein the AD-specific gene is selected from the group comprising ATF3, phosphodiesterase 4 B, CXCL2, BCL2 related protein A2, dual specificity phosphatase 2, TNFα-induced protein 3/A20, BTEB1, CXCL3, Chemokine CCL-3 like, CCL-4, CCL20, CX2CR1, amphiregulin, thrombomodulin, heparin binding EGF-like growth factor, DNA-damaged inducible transcript, V28 chemokine-like receptor, TRAIL, MAPK6, E4BP4, PBEF1, thrombospondin 1, MAFF, HSP70, CCL2, MCP-3, CCR2, CX3CR1, DOK1, HBB, G-gamma globin, THBD, PHLDA1, DTR, GNLY, and any combination thereof.

2. A method of diagnosing, screening for or predicting development of an inflammatory disease in a subject, said method comprising:
determining the level of from at least one to at least four inflammatory-specific gene products in a biological sample isolated from the subject wherein the inflammatory-specific gene is selected from the group comprising HSPC228, 34703_f_at, MCP-3, CCL2, EMP1, CDC42, TLE3, SPRY2, p40BBP, HSPC060, NAB2, HSPA1A, HSPA1B, MAPRE2, OAS1 and any combination thereof.

3. The method according to claim 2, wherein said inflammatory disease is an auto-inflammatory disease.

4. The method according to claim 2, wherein determining the level comprises determining the level of said at least one AD- or inflammatory-specific gene product in said isolated sample relative to the level of said at least one specific gene product in a control sample.

5. The method according to claim 2, wherein the level of said gene product is determined at the DNA, RNA level, and/or mRNA level.

6. The method according to claim 5, wherein determining the level is performed by contacting a nucleic acid extract of said sample with at least one probe comprising a sequence that hybridizes to a nucleotide region encoding an inflammatory-specific gene.

7. The method according to claim 6, wherein determining the level further comprises contacting the nucleic acid extract of said sample with at least one probe comprising a sequence that hybridizes to a nucleotide encoding a housekeeping gene.

8. The method according to claim 7, wherein said at least one nucleotide probe is immobilized on a solid support.

9. The method according to claim 6, wherein said nucleotide probes comprise DNA, RNA or cDNA and/or wherein said nucleic acid extract comprises nucleic acids with a detectable label.

10. The method according to claim 2, wherein the level of gene product is determined at the protein level.

11. The method according to claim 10, wherein determining the level is performed by contacting a protein extract of said sample with a specific binding partner of at least one protein encoded by an AD- or inflammatory- or specific gene under conditions that allow formation of a complex between said binding partner and said protein and detecting complex formation.

12. The method according to claim 1, wherein a subject is determined to have an increased probability of having bipolar disorder if the mRNA level of ATF3, Phosphodiesterase 4 B, CXCL2, BCL2 related protein A2, dual specificity phosphatase 2, TNFα-induced protein 3/A20, amphiregulin, thrombomodulin, heparin binding EGF-like growth factor, DNA-damaged inducible transcript, V28 chemokine-like receptor, MAPK6, E4BP4, PBEF1,thrombospondin 1, MAFF, HBB, G-gamma globin, THBD, PHLDA1, DTR and/or GNLY in a sample of the subject is at least two-fold higher and/or wherein the mRNA level of CX2CR1, TRAIL, HSP70, CCR2, CX3CR1 and/or DOK1 is at least two-fold lower compared to the presence of said mRNA in a sample of a healthy subject.

13. The method according to claim 1, wherein a subject is determined to have an increased probability of developing bipolar disorder if the mRNA level of amphiregulin, thrombomodulin, heparin binding EGF-like growth factor, DNA-damaged inducible transcript, V28 chemokine-like receptor and/or TRAIL in a sample of the subject is at least two-fold higher and/or wherein the mRNA level of ATF3, Phosphodiesterase 4 B, CXCL2, BCL2 related protein A2, Dual specificity phosphatase 2, TNFα-induced protein 3/A20, BTEB1, CXCL3, chemokine CCL-3 like, CCL-4, CCL20 and/or CX2CR1 is at least two-fold lower compared to the presence of said mRNA in a sample of a healthy subject.

14. The method according to claim 2, wherein a subject is determined to have an increased probability of having or developing an inflammatory disease if the mRNA level of HSPC228, 34703_f_at, MCP-3, CCL2, EMP1, CDC42, TLE3, SPRY2, p40BBP, HSPC060, and/or NAB2 in a sample of the subject is at least two-fold higher and/or wherein the mRNA level of HSPA1A, HSPA1B, MAPRE2 and/or OAS1 is at least two-fold lower compared to the presence of said mRNA in a sample of a healthy subject.

15. A kit for diagnosing or screening for affective disorder (AD) in a subject, said kit comprising at least one reagent specifically reactive with an AD-specific gene product selected from the group comprising ATF3, Phosphodiesterase 4 B, CXCL2, BCL2 related protein A2, Dual specificity phosphatase 2, TNFα induced protein 3/A20, BTEB1, CXCL3, Chemokine CCL-3 like, CCL-4, CCL20, CX2CR1, Amphiregulin, Thrombomodulin, Heparin binding EGF-like growth factor, DNA-damaged inducible transcript, V28 chemokine-like receptor, TRAIL, MAPK6, E4BP4, PBEF1, Thrombospondin 1, MAFF, HSP70, CCL2, MCP-3, CCR2, CX3CR1, DOK1, HBB, G-gamma globin, THBD, PHLDA1, DTR, GNLY, and any combination thereof.

16. A kit for diagnosing or screening for an inflammatory disease in a subject, comprising at least one reagent specifically reactive with an inflammatory-specific gene product selected from the group comprising HSPC228, 34703_f_at, MCP-3, CCL2, EMP1, CDC42, TLE3, SPRY2, p40BBP, HSPC060, NAB2, HSPA1A, HSPA1B, MAPRE2, OAS1, and any combination thereof.

17. The kit of claim 15, wherein said reagent comprises one of more antibodies, one or more antibody fragments, and/or one or more nucleotide probes.

18. The kit of claim 16, wherein said at least one reagent is immobilized on a solid support.

19. An array of nucleotide probes comprising at least 10 nucleotide bases in length, wherein at least one probe hybridizes to a fragment of at least one AD-specific gene selected from the group comprising ATF3, Phosphodiesterase 4 B, CXCL2, BCL2 related protein A2, Dual specificity phosphatase 2, TNFα-induced protein 3/A20, BTEB1, CXCL3, chemokine CCL-3 like, CCL-4, CCL20, CX2CR1, amphiregulin, thrombomodulin, heparin binding EGF-like growth factor, DNA-damaged inducible transcript, V28 chemokine-like receptor, TRAIL, MAPK6, E4BP4, PBEF1, thrombospondin 1, MAFF, HSP70, CCL2, MCP-3, CCR2, CX3CR1, DOK1, HBB, G-gamma globin, THBD, PHLDA1, DTR, GNLY, and any combination thereof.

20. The array of nucleotide probes of claim 19, wherein at least one probe hybridizes to a fragment of at least one housekeeping gene.

21. A method of diagnosing or screening for increased risk of developing an affective disorder in a subject, said method comprising:
analyzing a biological sample from the subject with the kit of claim 15 so as to diagnose or screen for increased risk of developing an affective disorder in the subject.

22. An array of nucleotide probes comprising at least 10 nucleotide bases in length, wherein at least one probe hybridizes to a fragment of at least one inflammatory-specific gene selected from the group comprising HSPC228, 34703_f_at, MCP-3, CCL2, EMP1, CDC42, TLE3, SPRY2, p40BBP, HSPC060, NAB2, HSPA1A, HSPA1B, MAPRE2, OAS1, and any combination thereof.

23. The array of nucleotide probes of claim 22, wherein at least one probe hybridizes to a fragment of at least one housekeeping gene.

24. The method according to claim 3, wherein said inflammatory disease is selected from the group consisting of diabetes mellitus Type 1, bipolar disorder, rheumatoid arthritis, multiple sclerosis, psoriasis, Sjógren's syndrome, thyroid disease, systemic lupus erythematosus, scleroderma, inflammatory bowel disease, and combinations thereof.

25. The method according to claim 4, wherein the control sample comprises a biological sample isolated from a healthy subject.

26. The method according to claim 1, wherein determining the level comprises determining the level of said at least one AD- or inflammatory-specific gene product in said isolated sample relative to the level of said at least one specific gene product in a control sample.

27. The method according to claim 1, wherein the level of said gene product is determined at the DNA level, RNA level and/or mRNA level.

28. The method according to claim 27, wherein determining the level is performed by contacting a nucleic acid extract of said sample with at least one probe comprising a sequence that hybridizes to a nucleotide region encoding an AD-specific gene.

29. The method according to claim 28, wherein determining the level further comprises contacting a nucleic acid extract of said sample with at least one probe comprising a sequence that hybridizes to a nucleotide encoding a housekeeping gene.

30. The method according to claim 27, wherein said at least one nucleotide probe is immobilized on a solid support.

31. The method according to claim 28, wherein said nucleotide probes comprise DNA, RNA or cDNA and/or wherein said nucleic acid extract comprises nucleic acids with a detectable label.

32. The method according to claim 1, wherein the level of said gene product is determined at the protein level.

33. The method according to claim 32, wherein determining the level is performed by contacting a protein extract of said sample with a specific binding partner of at least one protein encoded by an AD- or inflammatory- or specific gene under conditions that allow formation of a complex between said binding partner and said protein and detecting complex formation.

34. The method of claim 1 wherein the biological sample comprises blood monocytes.

35. The method of claim 2 wherein the biological sample comprises blood monocytes.

36. The kit of claim 15 further comprising at least one reagent specifically reactive with a housekeeping gene product and/or a defined amount of an AD-specific gene product.

37. The kit of claim 16 further comprising at least one reagent specifically reactive with a housekeeping gene product and/or a defined amount of an AD-specific gene product.

38. The kit of claim 18, wherein said solid support is selected from the group consisting of a glass solid support, nylon solid support, and nitrocellulose solid support.
